(19) United States
(12) Patent Application Publication
(10) Pub. No.: US 2002/0106399 A1
(43) Pub. Date: Aug. 8, 2002

(54) Antimicrobial wipe

(75) Inventor: Catherine Durden, Midland Park, NJ (US)

Correspondence Address: Charles N.J. Ruggiero, Esq., Ohlandt, Greeley, Ruggiero & Perle, L.L.P., 10th Floor, One Landmark Square, Stamford, CT, 06901-2682, US

(73) Assignee: Playtex Products, Inc.

(21) Appl. No.: 09/950,862

(22) Filed: Sep. 12, 2001

Related U.S. Application Data
(60) Provisional application No. 60/251,465, filed on Dec. 5, 2000.

(51) Int. Cl. A61K 9/70; B32B 27/12

(52) U.S. Cl. 424/443; 442/123

(57) ABSTRACT

Accordingly, the present invention provides a wipe impregnated with a composition having alcohol, preferably ethanol, in a concentration about 60% to about 95% of the total weight of the composition. In addition, the composition has one or more ingredients that mitigate against the unpleasant or unwanted effects of high-concentration alcohol.

This application claims the benefit of U.S. Provisional Application No. 60/251,465, filed Dec. 5, 2000, the contents of which are hereby incorporated by reference into this application.

### BACKGROUND OF THE INVENTION

1. Field of the Invention

The present invention generally relates to wipes impregnated with a liquid. More particularly, this invention relates to wipes impregnated with an antimicrobial liquid that has antibacterial and antiviral benefits, as well as kills germs and cleans skin, as well as surfaces.

2. Background of the Invention

Millions of germs share the environment with humans. A germ is generally defined as any microbial organism from which growth and development are expected. Thus, a germ may be a viruses, bacteria, mold, fungus, pollen, protozoan, yeast, and algae.

Many germs have the capacity to cause sickness and infection. Accordingly, controlling germs, such as viruses and bacteria, on human skin and inanimate surfaces deserves serious attention. In light of the foregoing, there is an ongoing need for products, such as wipes, that disinfect skin and inanimate surfaces.

A variety of chemicals may be used as antimicrobial agents. One such chemical is alcohol, which is lethal in high concentrations against the vegetative forms of many bacteria, as well as enveloped viruses. In fact, alcohols have a broad spectrum of virucidal activity, demonstrating effectiveness against lipophilic viruses (adenovirus, herpes, and influenza) as well as hydrophilic viruses (poliovirus 1, echovirus 6, and coxsackie B1). Alcohol in concentrations of 60 percent by weight of the total weight of the composition (wt. %) to 95 wt. % has been recognized as safe for over-the-counter daily topical use as an antimicrobial/antibacterial agent. However, when present in a high concentration, alcohols often have a distinctive and unpleasantly strong aroma. High concentration alcohol also tends to dry out the skin.

Accordingly, there is a need for products, such as wipes, that effectively disinfect human skin and inanimate surfaces by using alcohol in high concentration, yet are pleasant to use and do not adversely affect the look and feel of skin.

### SUMMARY OF THE INVENTION

It is an object of the present invention to provide an antimicrobial wipe for use on both human skin and inanimate surfaces.

It is another object of the present invention to provide such a wipe that is both antimicrobial and pleasing for consumers to use.

It is a further object of the present invention to provide such a wipe that has antibacterial and antiviral benefits.

It is still a further object of the present invention to provide such as wipe that kills germs and cleans skin, as well as surfaces.

Accordingly, the present invention provides a wipe impregnated with a composition having alcohol, preferably ethanol, in a concentration about 60% to about 95% of the total weight of the composition. The composition preferably also has a surfactant. In addition, the composition may have one or more ingredients that mitigate against the unpleasant or unwanted effects of high-concentration alcohol.

### DETAILED DESCRIPTION OF THE INVENTION

The present invention provides an antimicrobial composition having a high percentage of one of more alcohols that preferably is combined or impregnated into a wipe. Preferably, the antimicrobial wipe composition has a surfactant. In addition, the antimicrobial wipe composition may have one or more ingredients that militate against the unpleasant or unwanted effects of high-concentration alcohol. Still further, the antimicrobial wipe composition may have water.

The antimicrobial wipe cleans and disinfects both human skin and/or inanimate surfaces. The antimicrobial wipe is effective against or to control viruses, bacteria, and other harmful germs, such that the wipe is considered an antiviral wipe. It is also preferable that the wipe is designed for use both on human skin and on a wide variety of inanimate surfaces, such as countertops, drawers, shelves, and metal fixtures.

The antimicrobial composition according to the present invention is used in conjunction with a wipe, such as an absorbent and/or nonwoven sheet. It is preferable that the wipe is made of a substantially fibrous or semi-fibrous material, such as rayon, polyester, or cotton.

The wipe of the present invention has alcohol. The alcohol controls germs, such as M. tuberculosis and the vegetative forms of other bacteria, enveloped viruses, as well as lipophilic and hydrophilic viruses. Alcohols that can be used in the present invention include ethanol, propanol, and isopropanol. Ethanol is a preferred alcohol for use in the present invention.

The preferred concentration of alcohol is about 60 weight percent wt. %) to about 95 wt. % of the total weight of the wipe composition. The more preferred range of alcohol in the present invention is about 60 wt. % to about 80 wt. %. The most preferred amount of alcohol is about 65 wt. %.

As stated above, one or more surfactants or mild surfactants that are also low foaming surfactants. Surfactants include one or more amphoteric imidazoline derivatives, fatty sulfosuccinate esters and amides, ethoxylated sorbitan esters, alkylamido alkylamines, betaines, alkoxylated alcohols, phosphorus derivatives, quaternary ammonium compounds, ethoxylated alkyl phenols, alkyl ether sulfates, or any mixtures. Such preferred surfactants include disodium cocoamphodiacetate, cocomidopropyl betaine, and other surfactants based on coconut derivatives and/or betaines. When present, surfactants are preferably present in an amount about 0.01 wt. % to about 10 wt. % of the total weight of the composition. The more preferred range of surfactants is about 0.05 wt. % to about 5 wt. %, with about 0.5 wt. % being the most preferred concentration.

Preferably, the present invention has a topical vehicle for the alcohol and surfactant. The vehicle is formulated with ingredients to offset the undesirable qualities of high-concentration alcohol. These ingredients include one or more skin conditioners, preservatives, and mixtures thereof. Also, the vehicle may have water.

Water is preferably present in an amount about 5 wt. % to about 40 wt. %. More preferably water is present in an amount about 33 wt. %.

Skin conditioners useful in the present invention include lanolin and lanolin derivatives, polyols including glycerin and sorbitol, botanical derivatives including aloe, vitamins including vitamin E, silicones including dimethicone, and quaternary surfactants. The skin conditioners provide a pleasant after-feel on the skin.

When present, skin conditioners are preferably present in an amount about 0.01 wt. % to about 10 wt. %. The preferred amount of skin conditioners is about 0.05 wt. % to about 5 wt. %, with about 1 wt. % being the most preferred concentration. When used in a mixture with alcohol and one or more surfactants, as described below, the composition has about 60 wt. % to about 95 wt. % alcohol, about 0.01 wt. % to about 10 wt. % surfactants, and about 0.05 wt. % to about 5 wt. % skin conditioners. Preferably, the composition has about 60 wt. % to about 80 wt. % alcohol, about 0.05 wt. % to 5 wt. % surfactants, and about 0.05 wt. % to about 5 wt. % skin conditioners.

The present composition may have one or more preservatives. The preservatives acts to militate against unwanted mold or bacteria in the finished product. Preservatives useful in the present invention include parabens, organic acids and their derivatives, including sorbic acid and malic acid, iodophors, quaternary ammonium compounds, urea derivatives, isothiazolinones, alkyl substituted amino acids may be included in the composition. When present, preservatives are preferably present in an amount about 0.05 wt. % to about 2 wt. %. The more preferred range of preservatives is about 0.1 wt. % to about 1 wt. %, with 0.2 wt. % being the most preferred concentration.

When used in a mixture with alcohol, one or more surfactants and one or more skin conditioners, the composition has about 60 wt. % to about 80 wt. % alcohol, about 0.01 wt. % to about 10 wt. % surfactants, about 0.01 wt. % to about 10 wt. % skin conditioners, and about 0.05 wt. % to about 2 wt. % preservatives.

The present wipe composition may have other optional ingredients, such as pH adjusters and fragrances. When present, these optional ingredients are preferably present in an amount about 0.05 wt. % to about 2 wt. %. The more preferred range of optional ingredients is about 0.1 wt. % to about 1 wt. %, with 0.5 wt. % being the most preferred concentration.

In use, a wipe impregnated with an antimicrobial composition according to the present invention would preferably be used on human skin and/or inanimate surfaces to clean and control viruses, bacteria, and other harmful germs. Furthermore, depending upon the specific formulation, a composition according to the present invention may be used for other purposes, such as, for example, soothing.

The following are examples of an antimicrobial or antibacterial composition that may be used with a wipe.

EXAMPLE 1

| Ingredient | approximate wt. % |
|---|---|
| Water | 33.0 |
| Ethanol | 65.0 |
| Other ingredients including: | QS |
| coco-glucoside/glyceryl oleate, propylene | |
| glycol, glycerin, aloe vera gel, citric acid, | |
| and potassium sorbate | |
| | 100.00 |

EXAMPLE 2

| Ingredient | approximate wt. % |
|---|---|
| Water | 33.0 |
| Ethanol | 65.0 |
| Other ingredients including: | QS |
| PPG-12/PEG-50 lanolin, vitamin E | |
| acetate, propylene glycol, glycerin, | |
| aloe vera gel, citric acid, and | |
| potassium sorbate | |
| | 100.00 |

EXAMPLE 3

| Ingredient | approximate wt. % |
|---|---|
| Deionized water | 33.0 |
| Ethanol | 65.0 |
| Other ingredients including: | QS |
| disodium cocoamphodiacetate, | |
| dimethicone propylene glycol, | |
| glycerin, aloe vera gel, | |
| citric acid, and potassium sorbate | |
| | 100.00 |

The present invention having been described with particular reference to the preferred forms thereof, it will be obvious that various changes and modifications may be made herein without departing from the spirit and scope of the invention as defined in the appended claims.

What is claimed is:

1. A wipe impregnated with a topical antimicrobial composition comprising:
at least one alcohol being present in an amount about 60 percent to about 95 percent by weight of the total weight of said composition; and
a cleansing agent.

2. The wipe of claim 1, wherein said alcohol is selected from the group consisting of ethanol, propanol, isopropanol, and mixtures thereof.

3. The wipe of claim 1, wherein said cleansing agent is a surfactant.

4. The wipe of claim 3, wherein said surfactant is selected from the group consisting of phosphorus derivatives, ethoxylated alkyl phenol, quaternary ammonium compound, amphoteric imidazoline derivatives, fatty sulfosuccinate esters and amides, ethoxylated sorbitan esters, alkylamido alkylamines, betaines, alkoxylated alcohols, alkyl ether sulfates, amphoteric imidazoline derivatives, fatty sulfosuccinate esters and amides, ethoxylated sorbitan esters, alkylamido alkylamines, betaines, alkoxylated alcohol, alkyl ether sulfate, and mixtures thereof.

5. A wipe impregnated with a topical antimicrobial composition comprising:
at least one alcohol present in an amount about 60 percent to about 95 percent by weight of the total weight of said composition;
a cleansing agent; and
a skin conditioner.

6. The wipe of claim 5, wherein said alcohol is selected from the group consisting of ethanol, propanol, isopropanol, and mixtures thereof.

7. The wipe of claim 5, wherein said cleansing agent is a surfactant.

8. The wipe of claim 7, wherein said surfactant is selected from the group consisting of amphoteric imidazoline derivatives, fatty sulfosuccinate esters and amides, ethoxylated sorbitan esters, alkylamido alkylamines, betaines, alkoxylated alcohols, phosphorus derivatives, quaternary ammonium compounds, ethoxylated alkylphenols, alkyl ether sulfates, and mixtures thereof.

9. The wipe of claim 5, wherein said skin conditioner is selected from the group consisting of lanolin and lanolin derivatives, polyols, botanical derivatives, vitamins, silicones, quaternary surfactants, esters, sugars and mixtures thereof.

10. A wipe impregnated with a composition for topical application, said composition comprising:
one or more alcohols present in an amount about 60 percent to about 95 percent by weight of the total weight of said composition;
a cleansing agent; and
a preservative.

11. The wipe of claim 10, wherein said alcohols are selected from the group consisting of ethanol, propanol, isopropanol, and mixtures thereof.

12. The wipe of claim 10, wherein said cleansing agent is a surfactant.

13. The wipe of claim 12, wherein said surfactant is selected from the group consisting of amphoteric imidazoline derivatives, fatty sulfosuccinate esters and amides, ethoxylated sorbitan esters, alkylamido alkylamines, betaines, alkoxylated alcohols, phosphorus derivatives, quaternary ammonium compounds, ethoxylated alkylphenols, alkyl ether sulfates, and mixtures thereof.

14. The wipe of claim 10, wherein said preservative is selected from the group consisting of parabens, organic acids and their derivatives, iodophors, quaternary ammonium compounds, urea derivatives, isothiazolines, alkyl substituted amino acids and mixtures thereof.

15. A wipe impregnated with a composition for topical application, said composition comprising:
one or more alcohols selected from the group consisting of ethanol, propanol, isopropanol, and mixtures thereof present in an amount about 60 percent to about 95 percent by weight of the total weight of said composition;
a cleansing agent; and
a mitigating ingredient.

16. The wipe of claim 15, wherein said cleansing agent is a surfactant.

17. The wipe of claim 16, wherein said surfactant is selected from the group consisting of amphoteric imidazoline derivatives, fatty sulfosuccinate esters and amides, ethoxylated sorbitan esters, alkylamido alkylamines, betaines, alkoxylated alcohols, phosphorus derivatives, quaternary ammonium compounds, ethoxylated alkylphenols, alkyl ether sulfates, and mixtures it thereof.

18. The composition of claim 15, wherein said mitigating ingredient is a skin conditioner.

19. The composition of claim 15, wherein said mitigating ingredient is a preservative.

20. The composition of claim 15, wherein said mitigating ingredient comprises a skin conditioner and a preservative.

21. The composition of claim 15, wherein said mitigating ingredient is selected from the group consisting of lanolin and lanolin derivatives, polyols, botanical derivatives, vitamins, silicones, quaternary surfactants, parabens, organic acids and their derivatives, iodophors, quaternary ammonium compounds, urea derivatives, isothiazolines, alkyl substituted amino acids and mixtures thereof.

22. A method for cleaning and disinfecting human skin comprising the step of:
rubbing said human skin with a wipe impregnated with a composition having one or more alcohols present in an amount about 60 percent to about 95 percent by weight of the total weight of said composition; a cleansing agent; and one or more mitigating ingredients adapted to counteract the tendency of high-concentration alcohols to adversely affect the look and feel of skin.

23. The wipe of claim 22, wherein said cleansing agent is a surfactant.

24. The wipe of claim 23, wherein said surfactant is selected from the group consisting of amphoteric imidazoline derivatives, fatty sulfosuccinate esters and amides, ethoxylated sorbitan esters, alkylamido alkylamines, betaines, alkoxylated alcohols, phosphorus derivatives, quaternary ammonium compounds, ethoxylated alkylphenols, alkyl ether sulfates, and mixtures thereof.

25. The composition of claim 22, wherein said mitigating ingredient is selected from the group consisting of skin conditioners, preservatives, and mixtures thereof.

26. A method for cleaning and disinfecting an inanimate surface comprising the step of:
rubbing said inanimate surface with a wipe impregnated with a composition having one or more alcohols present in an amount about 60 percent to about 95 percent by weight of the total weight of said composition; a cleansing agent; and one or more mitigating ingredients.

27. The wipe of claim 26, wherein said cleansing agent is a surfactant.

28. The wipe of claim 27, wherein said surfactant is selected from the group consisting of amphoteric imidazoline derivatives, fatty sulfosuccinate esters and amides, ethoxylated sorbitan esters, alkylamido alkylamines, betaines, alkoxylated alcohols, phosphorus derivatives, quaternary ammonium compounds, ethoxylated alkylphenols, alkyl ether sulfates, and mixtures thereof.

29. The composition of claim 26, wherein said mitigating ingredient is selected from the group consisting of skin conditioners, preservatives, and mixtures thereof.
